(19) United States
(12) Patent Application Publication
(10) Pub. No.: US 2022/0218012 A1
(43) Pub. Date: Jul. 14, 2022

(54) FOOD COMPOSITION AND FOOD OR DRINK PRODUCT FOR SUPPRESSING ELEVATION OF SERUM REMNANT LIPOPROTEIN CHOLESTEROL LEVELS, AND SUPPRESSING METHOD

(71) Applicant: NISSIN FOODS HOLDINGS CO., LTD., Osaka-shi, Osaka (JP)

(72) Inventor: Nobuko INOIKE, Osaka (JP)

(21) Appl. No.: 17/605,772

(22) PCT Filed: Sep. 2, 2020
(86) PCT No.: PCT/JP2020/033225

§ 371 Date: Oct. 22, 2021

(30) Foreign Application Priority Data
Sep. 10, 2019 (JP) 2019-164275

(51) Int. Cl. A23L 33/21; A23L 2/52

(52) U.S. Cl. CPC A23L 33/21; A23V 2002/00; A23L 2/52

(57) ABSTRACT

[Problem]

To suppress the elevation of RLP-C levels caused by a meal in order to prevent atherosclerosis.

[Solution]

A food composition for suppressing elevation of postprandial serum remnant lipoprotein cholesterol levels, which comprises psyllium husk as an active ingredient. In addition, a method for suppressing elevation of postprandial serum remnant lipoprotein cholesterol levels, which comprises ingesting psyllium husk before or with a meal. As a result, it is possible to suppress the elevation of RLP-C levels resulting from a meal and to prevent atherosclerosis.

### TECHNICAL FIELD

The present invention relates to a food composition for suppressing elevation of postprandial serum remnant lipoprotein cholesterol levels. More specifically, the present invention relates to a composition for suppressing elevation of postprandial serum remnant lipoprotein cholesterol levels, which comprises psyllium husk as an active ingredient.

### BACKGROUND ART

Lifestyle-related diseases caused by lifestyle such as diet, exercise habits, smoking, and drinking have become a problem. For example, lifestyle-related diseases that develop due to diet include diabetes, obesity, hyperlipidemia, hypertension, colorectal cancer, and periodontal disease. Among these, in addition to having a large number of cases, the lifestyle-related diseases such as diabetes, obesity, hyperlipidemia, and hypertension are known to be closely related to the onset of arteriosclerosis, which causes cerebral infarction and myocardial infarction.

Arteriosclerosis can be classified into three main types: (i) the medial sclerosis type in which calcium accumulates in the media of arteries and calcifies; (ii) the atheroma type (atherosclerosis) in which lipids such as cholesterol accumulate in the lining of arteries to form a swelling called atheroma; and (iii) the arteriolosclerosis type in which arterioles are hardened. Of these, the atheroma form is considered to cause cerebral infarction and myocardial infarction.

In recent years, remnant lipoproteins (RLP-C) have received attention as proteins causing atherosclerosis. RLP-C is a general term for chylomicron (CM) remnants, VLDL remnants, or the like, which are intermediate metabolites of CMs, VLDL, or the like. Normally, CM and VLDL cannot invade vascular endothelium due to their large size, but small particles of RLP-C can invade vascular endothelium. Further, RLP-C having invaded vascular endothelium has been reported to be ingested by macrophages via the apoB48 receptor on macrophages, to promote the formation of foamy macrophages, and to form arteriosclerotic lesions (See Non-Patent Literature 1).

### CITATION LIST

### Non-Patent Literature

### Non-Patent Literature 1:

http://www.city.fukuoka.med.or.jp/kensa/ensinbunri/enshin_37_x.pdf

In order to prevent atherosclerosis, it is important to control serum RLP-C levels. However, one problem is that unlike other cholesterols, the serum RLP-C levels are easily affected by a meal and are thus difficult to control. Therefore, the suppression of the elevation of RLP-C levels caused by a meal had not yet been investigated.

### SUMMARY OF INVENTION

### Technical Problem

The present inventors investigated a substance capable of suppressing elevation of postprandial serum RLP-C levels. Then, the present inventors have newly found that the elevation of postprandial serum RLP-C levels can be suppressed by ingesting psyllium husk, and have completed the present invention.

### Solution to Problem

To solve the above problem, the present invention provides a food composition for suppresses elevation of postprandial serum remnant lipoprotein cholesterol levels, which comprises psyllium husk as an active ingredient, and a food or drink product using the food composition. In addition, to solve the above problem, the present invention provides a method for suppressing elevation of postprandial serum remnant lipoprotein cholesterol levels, which comprises ingesting psyllium husk before or with a meal.

According to such a configuration, elevation of postprandial serum RLP-C levels can be suppressed by ingesting a food composition comprising psyllium husk as an active ingredient.

### Advantageous Effects of Invention

According to the present invention, it is possible to suppress elevation of postprandial serum RLP-C levels. As a result, it is possible to prevent atherosclerosis.

### BRIEF DESCRIPTION OF DRAWING

FIG. 1 is a graph showing the time course of postprandial serum RLP-C levels when psyllium husk was ingested as compared with when a placebo food was ingested.

### DESCRIPTION OF EMBODIMENTS

Hereinafter, suitable embodiments of the present invention will be described.

<Psyllium Husk>

Psyllium husk is a dietary fiber material comprising polysaccharides with highly branched structures as main components. Examples of the psyllium husk used in the present invention include a husk obtained from the seeds of the plant Plantago ovata of the Plantaginaceae family, or a ground product thereof. Here, examples of the psyllium husk or a ground product thereof include those commercially available as psyllium, psyllium husk, psyllium husk powder, psyllium seed gum, Isagol® and the like (hereinafter, referred to as “psyllium husk powder”). In the present invention, a psyllium husk powder of any particle size and grade may be used, but the psyllium husk powder has preferably few impurities and a high purity.

The shape of the food composition of the present invention is not particularly limited. The above psyllium husk is highly safe, tasteless and odorless, and therefore it can be easily ingested continuously for a long period of time. Examples of the ingestion method include tablets, capsules, granules, powders, syrups, dry syrups, liquids, suspensions, jellies, and powdered beverages.

The food or drink product is not particularly limited, and examples thereof include beverages, spreads, dressings, breads, cooked rice, noodles, sauces, and confectionery.

The food or drink product of the present invention can further comprise various nutrients, various vitamins, minerals, dietary fiber, and a variety of additives.

### Example

Hereinafter, the present invention is described specifically based on an example. In the present example, an ingesting test was conducted on 76 healthy adults to examine the effect of the present invention.

<Preparation of Test Food>

As the test food, a beverage in which 3.6 g of psyllium was dissolved in 150 ml of water was used. In addition, a beverage containing no psyllium was used as the control food (placebo food). To maintain the blindness of the test, none of the beverages were distinguishable by appearance and flavor.

<Load Food>

180 g of hamburger, two butter-rolls, and 30 g of shoestring potatoes (total: 731 kcal, 24.4 g of proteins, 43.2 g of fats, and 61.3 g of carbohydrates) were used as load foods.

<Test>

In the present example, a randomized, double-blinded, crossover study on the test food and the placebo food was conducted. The washout period between the test food and the placebo food was 22 weeks. First, 76 subjects were randomized into two groups by a block-randomized method with sex, age, and fasting blood fat value at screening as the randomization factors. The general background of the subjects at screening was as shown in Table 1.

TABLE 1

| Parameter | Mean ± SE. |
|---|---|
| Age | 48.1 ± 1.1 |
| Height (cm) | 163.5 ± 1.0 |
| Body weight (kg) | 66.6 ± 1.3 |
| BMI | 24.8 ± 0.3 |
| Systolic Blood Pressure (mmHg) | 125.3 ± 1.8 |
| Diastolic Blood Pressure (mmHg) | 77.5 ± 1.3 |
| Total Cholesterol (mg/dL) | 210.4 ± 3.5 |
| LDL Cholesterol (mg/dL) | 131.2 ± 3.2 |
| HDL Cholesterol (mg/dL) | 62.6 ± 2.1 |
| Fasting Triglyceride (mg/dL) | 106.1 ± 2.2 |
| Fasting Glucose (mg/dL) | 86.3 ± 1.0 |
| HbA1c (%) | 5.3 ± 0.0 |
| Fasting Insulin (mU/mL) | 5.2 ± 0.3 |

Values are expressed as mean ± SE.
n = 76

The subjects completed their dinner by 21:00 on the day before each test day, and then fasted (only water allowed). Blood was collected on the following morning under fasting conditions, and then the test food or placebo food was ingested. Fifteen minutes after ingesting the test food or placebo food, high-fat load foods were ingested within 20 minutes, and blood was collected 2, 3, 4, and 6 h after the start of ingestion of the high-fat foods. In addition, the subjects fasted and rested in a sitting position until the blood collection 6 h after ingestion was completed.

<Blood Test and Analysis>

The levels of RLP-C contained in the blood were analyzed in a clinical laboratory using an existing method. The primary endpoints were the time course of RLP-C levels and the area under the blood level curve calculated by the trapezoidal method. Note that the test results are presented as the mean±standard error (SE).

Next, the obtained analysis results were statistically analyzed, and the results for when the test food was ingested were compared with the results for when the placebo food was ingested. Statistical analysis was performed by a two-tailed test, and the significance level was set to 5%. As the analysis software, SPSS for Windows (Ver. 24.0, manufactured by IBM Japan Ltd.) was used, and a generalized linear model (GLM) was selected as the statistical analysis method.

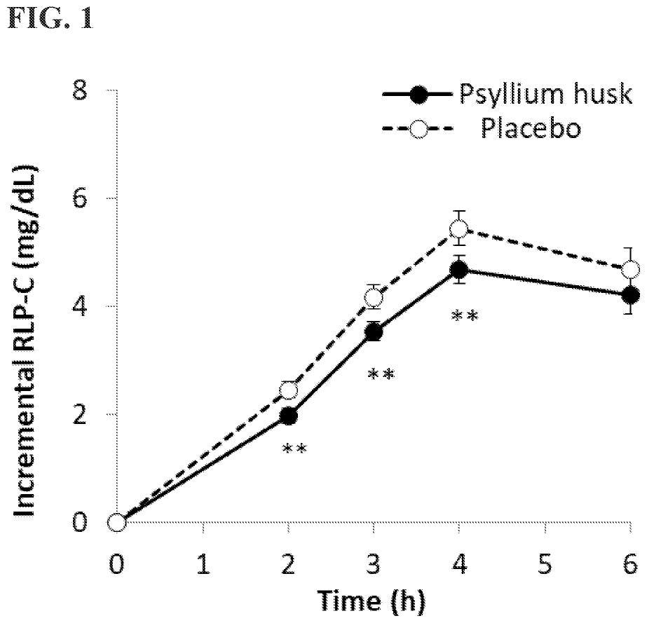

The results of the statistical analysis on the time course of the postprandial serum RLP-C levels are shown in FIG. 1.

As is clear from FIG. 1, the postprandial serum RLP-C levels for the test food are kept lower than the postprandial serum RLP-C levels for the placebo food. In particular, the value was significantly lower at 2, 3 and 4 h postprandial (P<0.01). In addition, the suppression of elevation of postprandial serum RLP-C levels began immediately after eating, and the difference from the placebo food was maximized at 4 h postprandial.

Next, using the data of FIG. 1, the area under the curve (AUC) of the blood levels up to 6 h postprandial was calculated by the trapezoidal method. AUC means the total amount of RLP-C postprandially absorbed in the blood. The results are shown in Table 2.

TABLE 2

| | | | Standard |
|---|---|---|---|
| Parameter | | Value | Deviation |
| RLP-C iAUC_{0-6 h} | Psyllium husk | 17.8 ± 1.0 | P < 0.003** |
| (mg · h/dL) | Placebo | 20.8 ± 1.2 | |

iAUC: incremental area under the curve.
Mean value was significantly different from placebo:
**p < 0.01, n = 76

As is clear from Table 2, the AUC of the test food showed a significantly lower value than the AUC of the placebo food (P<0.003). Specifically, it can be seen that the amount of RLP-C in the serum was reduced by about 15% by ingesting the test food as compared with the amount when the placebo food was ingested.

Here, the reason why psyllium husk, which is the test food, suppresses elevation of postprandial serum RLP-C levels is considered to be as follows. Psyllium husk has water-holding properties and forms a gel when dissolved in water. This psyllium gel is presumed to suppress the absorption of dietary lipids into the blood by preventing the contact of lipids with lipase in the gastrointestinal tract. Therefore, it is considered that reduction in the amount of lipids in the blood results in a decrease in postprandial serum RLP-C levels.

As described above, according to the present invention, elevation of postprandial serum RLP-C levels can be suppressed by the ingestion of psyllium husk. Since RLP-C is considered to be a substance causing atherosclerosis, it is considered that the development of atherosclerosis can be effectively suppressed by the suppression of RLP-C level elevation.

1. A food composition for suppressing elevation of postprandial serum remnant lipoprotein cholesterol levels, which comprises psyllium husk as an active ingredient.

2. A food or drink product, comprising the food composition for suppressing elevation of postprandial serum remnant lipoprotein cholesterol level according to claim 1.

3. A method for suppressing elevation of postprandial serum remnant lipoprotein cholesterol levels,
which comprises ingesting psyllium husk before or with a meal.

4. The method according to claim 3, wherein psyllium husk is ingested after being swollen with water.
